(19) United States
(12) Patent Application Publication
(10) Pub. No.: US 2021/0052258 A1
(43) Pub. Date: Feb. 25, 2021

(54) Multidirectional Device for Percutaneous Procedures

(71) Applicants: Behnoush Mortazavi Moghadam, Winnipeg (CA); Arman Rahimifar, Winnipeg (CA)

(72) Inventors: Behnoush Mortazavi Moghadam, Winnipeg (CA); Arman Rahimifar, Winnipeg (CA)

(21) Appl. No.: 16/548,872

(22) Filed: Aug. 23, 2019

(51) Int. Cl. A61B 10/02

(52) U.S. Cl. CPC A61B 10/0266; A61B 10/0283; A61B 2010/009; A61B 2010/0208; A61B 10/0275

(57) ABSTRACT

A multidirectional device for percutaneous procedures. The multidirectional device includes an operation chamber connected to a first end of a sheath via a hinge. A hinge system is operably connected to the operation chamber and allows for multidirectional movements of an operation apparatus disposed within the operation chamber. A housing is disposed on a second end of the sheath, wherein the housing includes controls for the hinge system. A main control system is operably connected to the housing to perform all hinge movement and housing functions and can be accessed remotely by a doctor performing the procedure. In some embodiments, the main control system is integrated into a CT or MRI scanner and is capable of being electronically navigated by artificial intelligence, convolutional neural networks, machine learning, autopilot navigation system, and the like. In other embodiments, a robot is used to control the movements of the multidirectional device.

### BACKGROUND OF THE INVENTION

The present invention relates to devices capable of performing percutaneous procedures. More specifically, the present invention relates to an automated, electronically navigated multidirectional device for performing percutaneous biopsy and interventional procedures.

Existing percutaneous devices used for performing biopsies and other interventional procedures include a penetrating needle that allows only for manual or semi-automated access to a target tissue, without offering any flexibility or ability in changing the direction of the needle tip once inside the skin. These devices lack the ability to change trajectory once beneath the skin. If the target tissue, deep within the body, is not aligned with the trajectory of the existing device, the target tissue will not be reached during the initial try of the procedure, causing unsuccessful, non-diagnostic result. To correct the misalignment, the entirety of the penetrating needle must be removed from the skin and the process re-attempted with no guarantee that further attempts will be more successful. Unfortunately, the attempts will continue to be performed on the patient until proper trajectory is achieved by reaching the target tissue with the penetrating needle, thereby significantly increasing the procedure time. This is especially disadvantageous for ill patients and children who are unable to hold still in a certain position for an extended period of time.

Additionally, due to the lack of complete automation of these percutaneous devices, healthcare professionals are required to be physically present with the patient during a procedure. For example, percutaneous biopsy of tissue is a well-accepted alternative to open surgical biopsy with needle localization for the determination of abnormal lesions seen by radiology images or ultrasound but not able to be felt by the surgeon. Biopsy techniques can vary depending upon the location and the size of the abnormal lesion. In general, these techniques often involve a radiology doctor who introduces a needle into the target lesion by puncturing the outer skin while watching the imaging equipment such as a computed tomography (CT) scanner. When the CT scanner is used the doctor typically is required to enter and exit the biopsy room during the biopsy procedure to avoid excessive radiation exposure. Although needle localization can be done by a computer and mechanical engineering system, it is still a partially blind biopsy as the radiologist cannot see the CT monitor when he or she has to obtain the sample tissue in the last moment of the biopsy procedure. The doctor needs to go back to the biopsy room again and pull out the stylet and manipulate the needle to cut the tissue sample in person without being able to watch the CT monitor.

Further, if the biopsy needles have a spring propulsion system, the doctor must release the trigger, which can cause pain, discomfort and inaccurate sampling by pushing or shaking the biopsy needles and patient's body. Another disadvantage is when the forced forward movement of the needle by the spring mechanism can push the needle too far forward to miss the target tissue. This can result in inaccurate sampling that requires repeated biopsy procedures that increase the pain and complications associated with repeated biopsy procedures. The rate of inaccurate or unsuccessful biopsies can be as high as 25% of total biopsy procedures.

In view of the above concerns, there is a need to provide a device for performing percutaneous procedures having an operation chamber that allows for an operation apparatus to be pivoted once in the body of a patient in order to prevent the complete removal of the operation apparatus if there is initial misalignment with the target tissue. This will allow for optimum precision and accuracy for the biopsy sampling and other results of percutaneous interventional procedures, which will not only significantly reduce the procedure time, but will reduce the need for anesthesia, conscious sedation and relaxing medications and increase patient comfort. Further, non-diagnostic results and unsuccessful attempts as well as complications such as post procedure bleeding and injury to the adjacent organs will be greatly decreased, and patient care, patient safety and satisfaction will be achieved.

In addition, there is a need for a device to be fully automated and capable of performing biopsy and other procedures remotely without requiring the doctor to travel to the patient's facility or transferring the patients for the purpose of biopsy and other percutaneous procedures. This will significantly improve workflow of the required healthcare providers, such as a radiology doctors, provide better access to highly skilled doctors, and decrease the cost of the image-guided percutaneous biopsy and procedures through reducing travel time to patient locations. In addition, the improved invention will decrease radiologists' and imaging personnel's radiation dose and hazardous exposures to bodily fluids and sharp needle tips, as well as decrease patients' radiation exposures.

In light of the devices disclosed in the known art, it is submitted that the present invention substantially diverges in design elements and methods from the known art and consequently it is clear that there is a need in the art for an improvement for a multidirectional device for percutaneous procedures. In this regard the instant invention substantially fulfills these needs.

### SUMMARY OF THE INVENTION

In view of the foregoing disadvantages inherent in the known types of multidirectional devices now present in the known art, the present invention provides a new multidirectional device wherein the same can be utilized for performing automated percutaneous biopsy and interventional procedures.

It is an objective of the present invention to provide a multidirectional device having an operation chamber connected to a first end of a sheath via a hinge. A hinge system is operably connected to the operation chamber and allows for multidirectional movements of the operation chamber and an operation apparatus disposed therein. A housing is disposed on a second end of the sheath, wherein the housing includes controls for the hinge system. A main control system is operably connected to the housing in order to perform all hinge movement and housing functions, wherein the main control system can be accessed remotely by a doctor performing the procedure.

It is another objective of the present invention wherein the hinge system comprises a ball disposed on the operation chamber and a socket disposed on the sheath allowing rotational movement therebetween. At least one cord extends from the ball, passing through a socket aperture and traversing along the sheath. The cord is controlled by at least one control on the housing that allows for incremental adjustment of the hinge along three axes and six degrees of freedom until the operation apparatus is aligned with the target tissue or site.

Yet another objective of the present invention wherein the main control system is integrated into a CT or MRI scanner and is capable of being electronically navigated by artificial intelligence, convolutional neural networks, machine learning, autopilot navigation system, and the like.

In other embodiments, the present invention includes a robot connectable to the main control system and used to control the movements of the multidirectional device.

It is therefore an object of the present invention to provide a new and improved multidirectional device for percutaneous procedures that has all of the advantages of the known art and none of the disadvantages.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of an embodiment of the multidirectional device for percutaneous procedures.

FIG. 2 shows an exploded view of the working end of an embodiment of the multidirectional device for percutaneous procedures.

FIG. 3 shows a cross sectional view of the hinge system between the operation chamber and the sheath of an embodiment of the multidirectional device for percutaneous procedures.

FIG. 4 shows a cross sectional view of the working end of an embodiment of the multidirectional device for percutaneous procedures, wherein the operation apparatus is a biopsy apparatus.

FIG. 5 shows a cross sectional view of the ball and socket connection of an embodiment of the multidirectional device for percutaneous procedures taken along line 5-5 of FIG. 4.

FIG. 6 shows a transparent view of the housing of an embodiment of the multidirectional device for percutaneous procedures.

FIG. 7 shows a perspective view of the housing of a first alternate embodiment of the multidirectional device for percutaneous procedures.

FIG. 8 shows a cross sectional view of the working end of a first alternate embodiment of the multidirectional device for percutaneous procedures.

FIG. 9 shows a perspective view of a second alternate embodiment of the multidirectional device for percutaneous procedures.

FIG. 10 shows a block diagram of an embodiment of the multidirectional device for percutaneous procedures.

### DETAILED DESCRIPTION OF THE INVENTION

Reference is made herein to the attached drawings. Like reference numerals are used throughout the drawings to depict like or similar elements of the multidirectional device. For the purposes of presenting a brief and clear description of the present invention, the preferred embodiment will be discussed as used for performing automated and electronically navigated percutaneous biopsy and interventional procedures. The figures are intended for representative purposes only and should not be considered to be limiting in any respect.

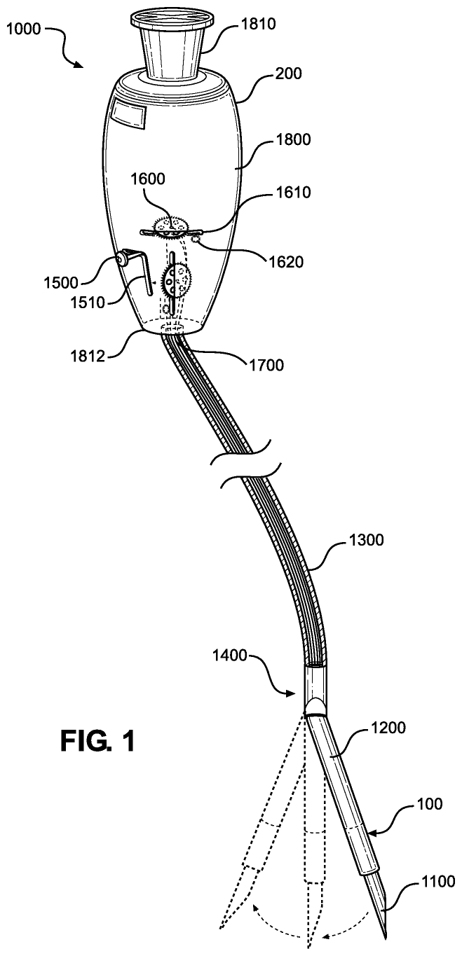

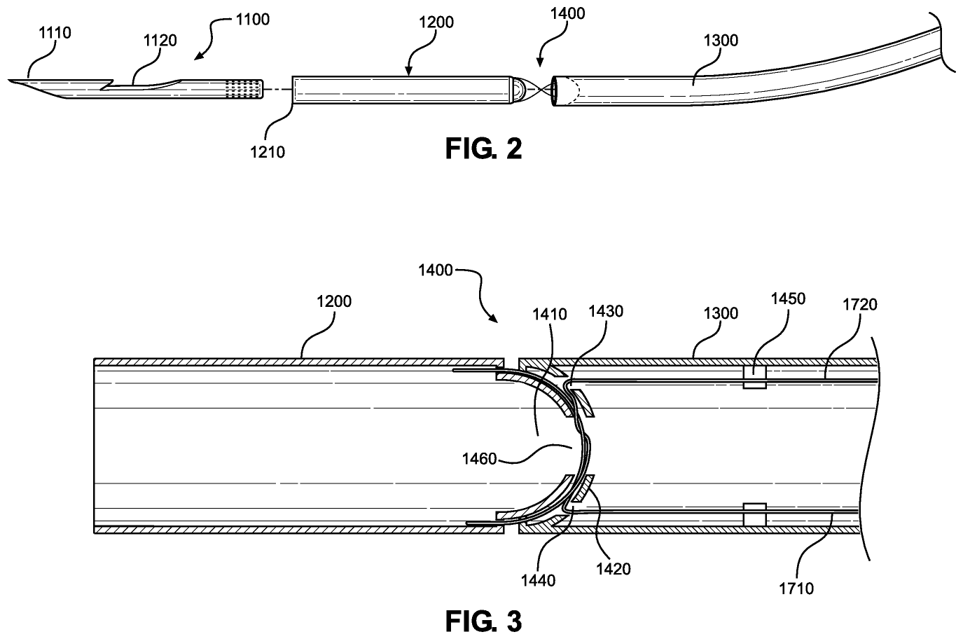

Referring now to FIGS. 1 and 2, there is shown a perspective view of an embodiment of the multidirectional device for percutaneous procedures and an exploded view of the working end of an embodiment of the multidirectional device for percutaneous procedures, respectively. The multidirectional device 1000 comprises a working end 100 having an operation chamber 1200 pivotally connected to a first end of a sheath 1300 via a pivot point or hinge 1400 and a housing 1800 disposed on an opposing second end 1812 of the sheath 1300. The hinge 1400 is configured to allow the operation chamber 1200 to rotate along three axes, with six degrees of freedom. An operation apparatus 1100 is housed within the operation chamber 1200 to allow for cooperative multidirectional movement with the operation chamber 1200. In use, the operation apparatus 1100, the operation chamber 1200, and a portion of the sheath 1300 are inserted into a patient and directed to a target site, such as a tissue to be biopsied. The operation chamber 1200 is adapted to rotate and incrementally adjust the direction of the operation apparatus 1100 while traveling within a body to a target site. This will reduce the need to remove the operation apparatus 1100 during a percutaneous procedure if the operation apparatus and chamber 1100, 1200 are misaligned with the target site. In turn, this improves the efficiency, precision and safety of the percutaneous procedure.

In the illustrated embodiment, the operation chamber 1200 comprises a hollow, cylindrical body having an open first end 1210 configured to receive the operation apparatus 1100 therein. The hinge 1400 is disposed on an opposing second end 1210 of the operation chamber 1200. In the illustrated embodiment, the hinge 1400 is a ball and socket joint, whereby the ball is disposed on the operation chamber 1200. The operation chamber 1200 allows for different types of operation apparatuses to be received therein for performing various percutaneous procedures, including but not limited to the following procedures: percutaneous image-guided biopsy, percutaneous image-guided endovascular procedures such as TIPS (Transjugular Intrahepatic Portosystemic Shunt), percutaneous image-guided intraluminal procedures such as nephrostomy, biliary drain insertion and abscess drain insertion, percutaneous image-guided radiofrequency ablation, percutaneous image-guided fiducial marker placement, percutaneous image-guided brachytherapy seeds placement, tissue electrocautery, tissue injection and aspiration. In the illustrated embodiment, the operation apparatus 1100 is removably secured to the operation chamber 1200 by any suitable fastener, including but not limited to a friction fit or a threaded connection.

In the illustrated embodiment, the operation apparatus 1100 is a biopsy apparatus comprising a cutting blade 1110 and a biopsy reservoir 1120, wherein the cutting blade 1110 is disposed at the distal tip of thereof and the biopsy reservoir 1120 is disposed on an opposing end of the operation apparatus 1100. The cutting blade 1110 is configured to cut a piece from the target tissue during backward movement of the operation apparatus 1100, whereas the biopsy reservoir 1120 receives the target tissue once it is cut and temporarily stores the tissue therein.

The multidirectional device 1000 comprises a hinge system, including the hinge 1400, that allows for automated and electronic control of the rotational movement of the hinge 1400 during percutaneous procedures. In the illustrated embodiment, the hinge system comprises a cord 1700 extending from the hinge 1400 and through the sheath 1300 and to a hinge control 1600. The cord 1700 is operably connected to the hinge control 1600, wherein the hinge control 1600 is disposed in the housing 1800. In the illustrated embodiment, the hinge control 1600 is a cogwheel scroll disposed within a cogwheel groove 1610, wherein the cord 1700 is connected to an axel thereof. In the illustrated embodiment, a cord spring is operably connected to the axel and provides tension to the cord 1700 such that when the cogwheel scroll is rotated, the cord 1700 is moved accordingly. Thus, the hinge control 1600 controls the movement of the operation apparatus and chamber 1100, 1200 at the hinge 1400. The cogwheel groove 1600 is disposed on the housing 1800 such that a portion of the cogwheel scroll is positioned within the housing and the remaining portion is positioned along the exterior of the housing in order to allow the cogwheel to be accessible by a user, while protecting the axel and the cord 1700 within the housing 1800. The shown embodiment further comprises a cogwheel brake 1620 configured to maintain a desired position of the cogwheel scroll. The cogwheel brake comprises a smaller cogwheel and a cogwheel spring positioned against and meshed with the cogwheel scroll, wherein the cogwheel brake is adapted to provide resistance against rotation of the cogwheel scroll.

In the illustrated embodiment, the housing 1800 serves as a handle and comprises a circular cross section. A middle section of the housing tapers outward providing a generally oblong shape. The housing 1800 comprises an actuation mechanism for driving the operation apparatus forward via an operation lever.

Referring now to FIG. 3, there is shown a cross sectional view of the hinge system between the operation chamber and the sheath of an embodiment of the multidirectional device for percutaneous procedures. In the illustrated embodiment, the hinge 1400 is a ball 1410 and socket 1420 joint. However, in alternate embodiments, the hinge 1400 comprises any suitable structure configured to provide rotational and incremental movements between the operation chamber 1200 and the sheath 1300. As references herein, “ball and socket” joint refers to a rounded member (“ball”) that fits into a cup-like depression (“socket”) and provides rotation about a common center. In some embodiments, the ball is disposed within the socket past a diameter of the ball. In other embodiments, the ball is partially disposed within the socket, so the diameter of the ball is outside the socket. In the shown embodiment, the ball is a hemispherical ball. The ball 1410 and socket 1420 each comprise an aperture 1460 aligned with one another forming ball and socket apertures. In the illustrated embodiment, the apertures 1460 are centrally aligned such that the apertures 1460 are concentric with the exterior of the operation chamber 1200 and sheath 1300, respectively. In the shown embodiment, the outer diameter of the sheath 1300 is equal to the outer diameter of the operation chamber 1200. In this way, as the operation chamber and sheath 1200, 1300 move smoothly through the patient.

In the illustrated embodiment, the hinge system comprises a first 1710, second 1720, third, and fourth cord, each connected to and evenly distributed about the ball 1410 (as seen in FIG. 5). Each cord is fastened to the ball 1410 and passes through a distinct socket cord aperture in the socket 1420 disposed on an opposing lateral side thereof, and traverses along the sheath 1300 until it reaches the designated axel of the cogwheel scroll. At the hinge 1400, the cords 1710, 1720 are coupled with the ball 1410 on the opposing side such that when tension is applied to the cord 1710, 1720 the ball 1410 rotates toward the cord 1710, 1720. The position of the cords 1710, 1720 at fixed intervals about the hinge 1400 allow for precise movement of the ball 1410, and consequently the operation chamber 1200 and the operation apparatus 1100. In the illustrated embodiment, the first cord 1710 passes through a first socket cord aperture 1430 and the second cord 1720 passes through a second socket cord aperture 1440, such that the first cord originates from an opposing side of the first socket cord aperture 1430 and the second cord 1720 originates from an opposing side of the second socket cord aperture 1440. The same is true for the third and fourth cords and apertures (not shown), but on opposing sides of the first and second cords and socket cord apertures. A cord harness 1450 is positioned along an interior side of the sheath 1300, securing each cord in position to prevent cord movement and tangling. In the illustrated embodiment, the cord harness 1450 is a tab that receives the cord therethrough and presses the received portion against the interior side of the sheath 1300. In this way, the interior of the sheath 1300 remains open to receive other components therethrough without cord obstructed.

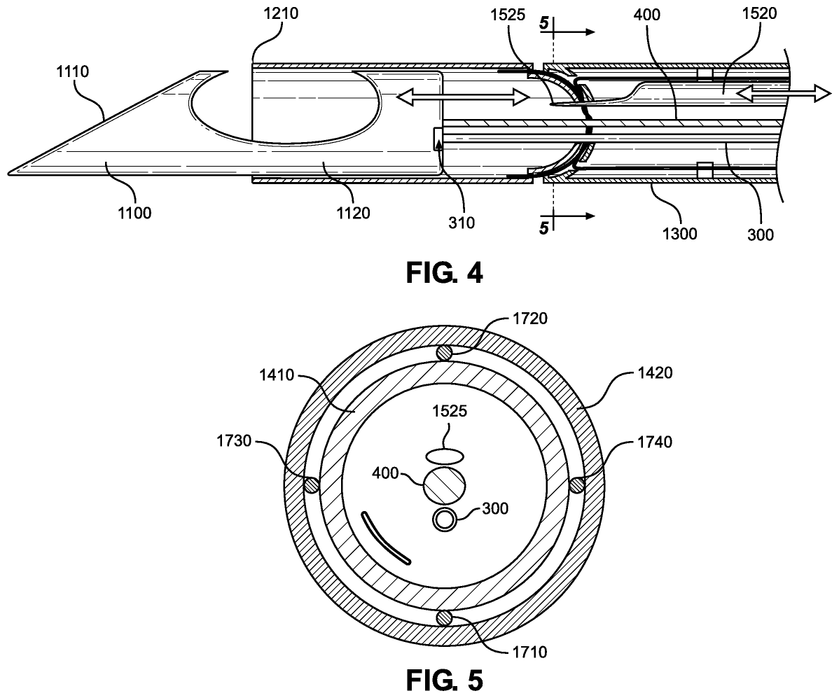

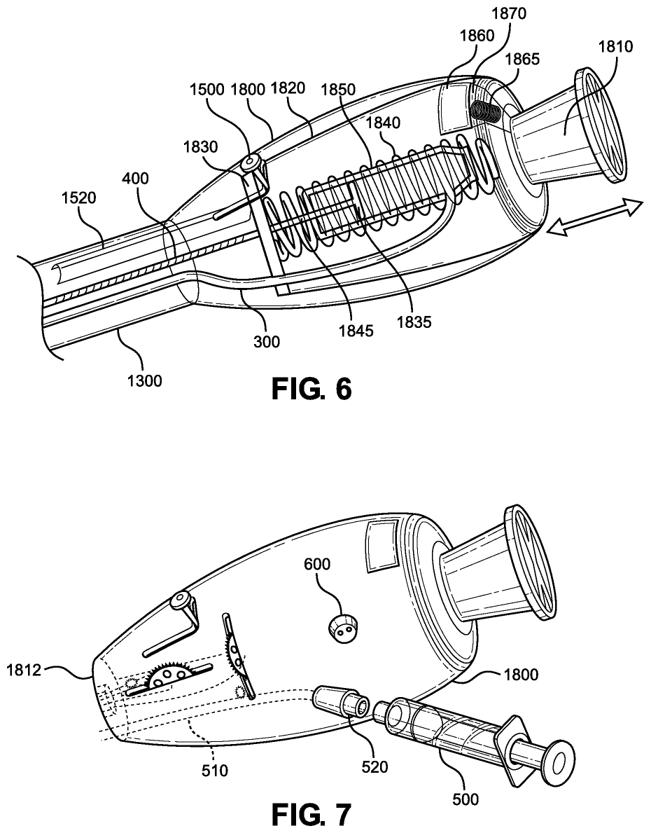

Now referring to FIGS. 4 and 5, there is shown a cross sectional view of the working end of an embodiment of the multidirectional device for percutaneous procedures, wherein the operation apparatus is a biopsy apparatus and a cross sectional view of the ball and socket joint of an embodiment of the multidirectional device for percutaneous procedures taken along line 5-5 of FIG. 4, respectively. The ball and socket apertures 1460 are configured to receive components extending from the housing, through the sheath 1300 and to the operation apparatus 1100. In the illustrated embodiment, a vacuum tube 300, the operation lever 400, and a hinge lock 1520 each pass through the ball and socket apertures 1460. In the shown embodiment, the vacuum tube 300 extends through the sheath 1300 from the housing (as seen in FIG. 6) and terminates at the biopsy reservoir 1120 of the operation apparatus 1100. A vacuum source provides suction through the vacuum tube 300 to the biopsy reservoir 1130 in order to displace a sampled tissue, from the target tissue, away from the cutting blade 1110 and into the biopsy reservoir 1120. Placing the sampled tissue into the biopsy reservoir 1120 prepares the operation apparatus 1100 for a next actuation. In the illustrated embodiment, a filter 310 is positioned at the opening of the vacuum tube 300 to prevent the sample tissue from leaving the biopsy reservoir 1120.

In the illustrated embodiment, the multidirectional device comprises a hinge lock system that prevents the hinge from rotating. The hinge lock system includes the hinge lock 1520 and a hinge release button (FIG. 1, 1500), wherein the hinge lock 1520 is configured to physically couple the ball 1410 and socket 1420 so as to prevent relative rotation about the hinge. The hinge release button is disposed within an L-shaped track 1510 on the housing and configured to move the hinge lock 1520 between a locked and unlocked position. In the shown embodiment, the hinge lock 1520 is a curved member that extends from the hinge release button at a first end, through the sheath 1300, and terminates at the hinge. The terminating end of the hinge lock 1520 comprises a flexible tip 1525 configured to penetrate the ball and socket aperture. The hinge release button is movable along the track in order to slidably move the hinge lock longitudinally along the sheath. In the unlocked position, the hinge release button is positioned furthest from the working end and the flexible tip 1525 is removed from the ball and socket aperture in order to allow the hinge to move. As the hinge release button moves along the track, closer to the working end, the flexible tip is reinserted within the ball and socket aperture and into the locked position. A hinge release button spring disposed in the housing, connected to the hinge release button, biases the hinge release button towards the locked position, whereas the L-shaped track stabilizes the hinge release button within a horizontal portion of the L-shaped track once hinge lock is released so as to prevent movement of the button along the track. In the illustrated embodiment the hinge lock 1520 is composed of a thin metal material.

The operation lever 400 extends from the operation apparatus 1100, which is the biopsy apparatus in the illustrated embodiment, to an actuation mechanism disposed in the housing. The operation lever 400 is movable longitudinally along the sheath 1300 so at push the operation apparatus 1100 outward from the open end 1210 of the operation chamber 1200. In the illustrated embodiment, the operation lever 400 is secured to a middle area of the operation apparatus 1100 such that the operation lever 400 is concentric therewith. The operation lever 400 comprises a non-bending, mildly flexible wire.

Referring now to FIG. 6, there is shown a transparent view of the housing of an embodiment of the multidirectional device for percutaneous procedures. In the illustrated embodiment, the housing 1800 is composed of plastic and houses the hinge controls, the hinge lock release button 1500, and at least one system to accompany the operation apparatus, including but not limited to the biopsy apparatus, a vascular access and wire placement system, an intraluminal access system and wire placement apparatus, a vacuum system, an injection apparatus, an electrocautery apparatus, a fiducial and brachytherapy marker placement apparatus, a radiofrequency ablation apparatus, an aspiration system and drainage apparatus, and a fiberoptic visualization apparatus.

The housing 1800 includes the actuation mechanism that initiates movement of the operation lever 400 to drive the operation apparatus to the desired target within a patient. In the illustrated embodiment, the actuation mechanism includes a push button 1810 disposed at the end of the housing 1800, a push assembly 1820 operably connected to the push button and the operation lever 400. The push assembly 1820 includes a base 1830, a housing spring 1840 and a cylinder 1850. As the push button 1810 is depressed, the housing spring 1840 compresses and the operation lever 400 is longitudinally moved towards the working end of the multidirectional device, thereby pushing the operation apparatus forward and away from the operation chamber. The housing spring 1840 is biased so as to force the push button 1810 in an undepressed position when the push button is released. A cam 1845 is fixed to the base 1830 and operably connected to a piston 1835, wherein the piston 1835 is disposed within the cylinder 1850.

A push button lock, accessible from the exterior of the housing 1800, is operably connected to the push button 1810 in order to prevent movement of the push button 1810. The push button lock is configured to ensure the push button has moved the operation lever entirely forward so as to allow the operation apparatus to reach the target tissue by locking the push button 1810 in the depressed position until a user is ready to release the push button. In the illustrated embodiment, the push button lock includes a lock button 1860 and a lock spring 1865 disposed in a slot 1870 through an upper end of the housing 1800. The lock spring 1865 forces the lock button 1860 to maintain the position of push button lock until the lock button 1860 is actuated.

In the illustrated embodiment, a vacuum system is disposed within the housing 1800 and operably connected to the vacuum tube 300. The vacuum system includes the base 1830, the cam 1845, and the piston 1835 operably connected to the cylinder 1850, wherein the vacuum tube 300 is in fluid communication with an interior of the cylinder 1850. The connection between the cylinder and the piston is airtight, with the vacuum tube 300 being the sole fluid pathway. In the illustrated embodiment, as the push button 1810 is depressed, the piston 1835 remains static while the cylinder 1850 slides towards the working end of the multidirectional device. As the push button 1810 is released, an internal volume formed by the cylinder 1850 and the piston 1835 increases, thereby creating a vacuum (negative pressure) within the cylinder 1850. As the push button 1810 is released during the percutaneous procedure, the suction is applied to the operation apparatus in order to displace the sampled tissue away from the cutting blade and into the biopsy reservoir. Once the sampled tissue is disposed in the biopsy reservoir, the operation apparatus is prepared for a next actuation. In some embodiments, the vacuum tube is operably connected to an external vacuum source.

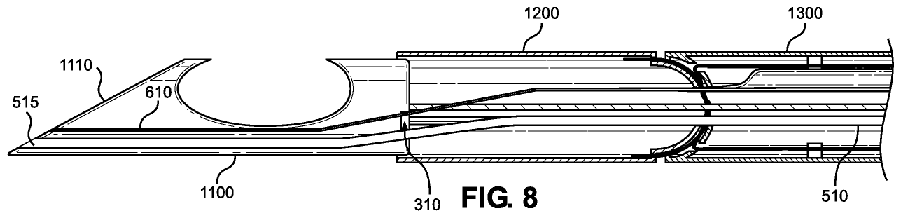

Referring now to FIGS. 7 and 8, there is shown a perspective view of the housing of a first alternate embodiment of the multidirectional device for percutaneous procedures and a cross sectional view of the working end of a first alternate embodiment of the multidirectional device for percutaneous procedures, respectively. In the illustrated embodiment, the multidirectional device for percutaneous procedures comprises an injection system and an electrocautery system. The injection system includes an injection hub 520 on the housing 1800, connected to an injection tube 510 traversing the length of the sheath 1300 and extending into the operation apparatus 1100. In the illustrated embodiment, the operation apparatus 1100 comprises an opening 515 on the tip 1110 thereof that allows for fluid, such as local anesthetic injections, gel foam injections, air dissecting, or saline dissecting, to be introduced to the patient at the beginning of the percutaneous procedure or at the end of the procedure. In the illustrated embodiment, the fluid is introduced through a syringe 500 injection at the injection hub 520 and then travels through the injection tube 510 and out of the opening 515 of the tip of the operation apparatus to a target site.

The electrocautery system comprises a plug or electrical connector 600 disposed on the housing 1800 and an electrode wire 610 extending along the length of the sheath 1300 and through the operation apparatus 1100. The electrode wire 610 terminates at the tip 1110 of the operation apparatus. The electrode wire 610 is configured to heat the tip 1110 in order to cauterize tissue during a percutaneous procedure. In the illustrated embodiment, the electrocautery system is controlled directly from the housing, however, in alternate embodiments the system is controlled remotely.

In some embodiments, the multidirectional device comprises a tissue transfer container including a bottle having an open upper end and a spout having a spout opening. The open upper end and spout opening are each coverable with a cap. A curved handle extends from the bottle and is configured to stabilize the container during tissue retrieval and minimize physician exposure to any sharp needle tip disposed on the operation apparatus.

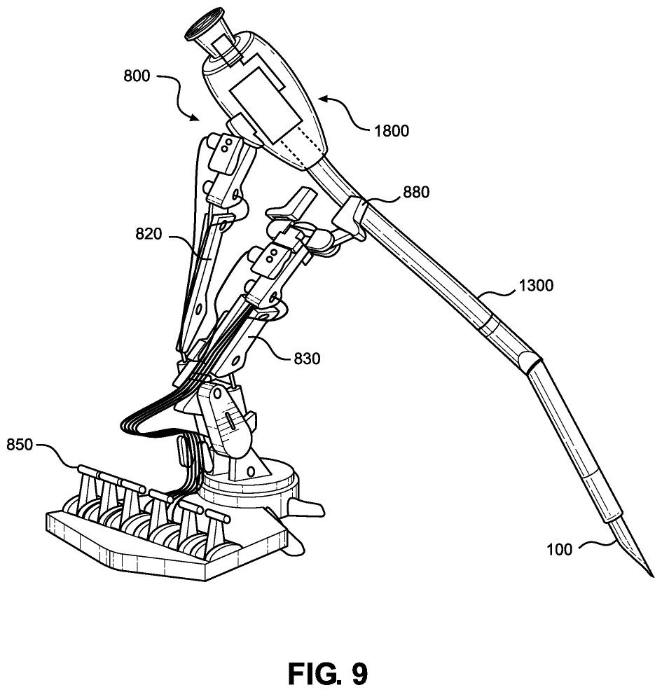

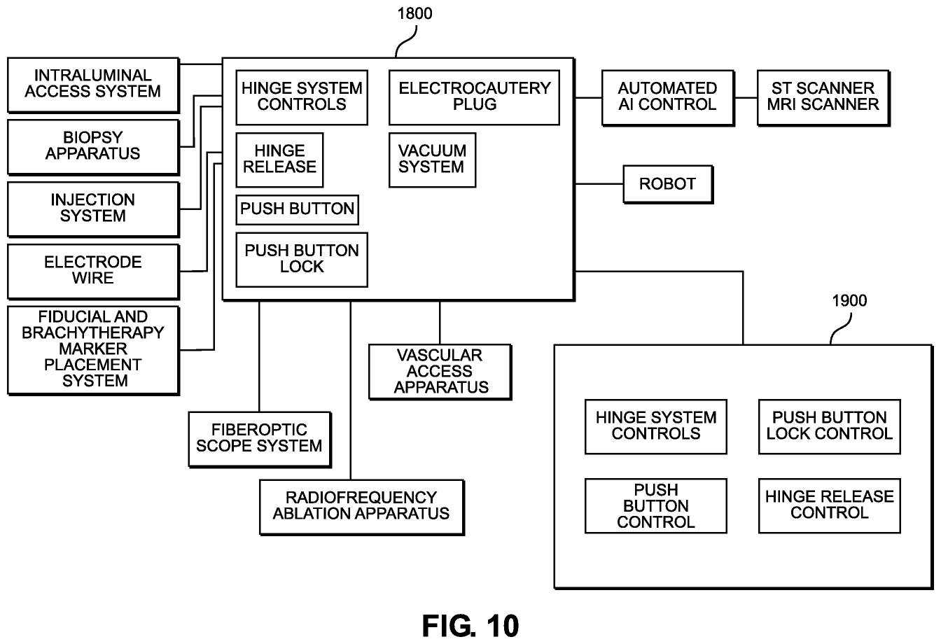

Referring now to FIGS. 9 and 10, there is shown a perspective view of a second alternate embodiment of the multidirectional device for percutaneous procedures and a block diagram of an embodiment of the multidirectional device for percutaneous procedures, respectively. A main control system is operably connected to the housing 1800 in order to perform all hinge movement and housing functions, wherein the main control system can be accessed remotely by a doctor performing the procedure. In some embodiments, the main control system is integrated into a CT or MRI scanner and is capable of being electronically navigated by artificial intelligence, convolutional neural networks, machine learning, autopilot navigation system, and the like. In other embodiments, a robotic arm is used to control the movements of the multidirectional device.

In some embodiments, the multidirectional device comprises a remote-control system. In one embodiment, the remote-control system is a hand-held controller 1900 device having a joystick operably connected to the main control system and configured to remotely operate the hinge system, the actuation mechanism, and any other system within the housing.

In some embodiments, the multidirectional device is operably connected to a robot 800 having a robotic arm 830 configured to advance the working end 100 of the multidirectional device into the patient. The robot 800 is configured to navigate the operation apparatus into the target tissue. In the illustrated embodiment, the robot has a five-axis robot arm 830 and an end-effector 880. The end-effector 880 is attached to the distal end of the robot arm 830 for the installation and insertion of the operation apparatus. A control system 850 is disposed on the robot to allow a physician to manipulate the robot 800. In some embodiments, the robot 800 includes a second arm 820 for connecting to the control functions of the housing 1800. In this way, the control system 850 of the robot 800 may be remotely accessed through the direct connection to the housing. In one example, when the procedure is to be performed with the aid of a computed tomography (CT) scanner, the control system is remotely located from the robot 800. This reduces the number of trips by the doctor into the biopsy room during the biopsy procedure reducing the radiation exposure. In some embodiments, the robotic arm is configured to be mounted and secured to the body of the patient via a fastener, such as adhesive tape. In other embodiments, the multidirectional device can be controlled from a bedside of the patient, from a CT or MRI control room, or remotely from longer distances. Alternatively, in some embodiments, the control system of the multidirectional device comprises is configured to perform percutaneous biopsy under direction of ultrasound or fluoroscopic guidance.

In one embodiment, the multidirectional device comprises an automated artificial intelligence control configured to integrate into the CT or MRI Scanner monitor and uses artificial intelligence, machine learning, convolutional neural networks and autopilot navigation to control the trajectory of the operation apparatus or follow a pre-defined trajectory determined by a performing radiologist. The automated artificial intelligence control is configured to determine pathway information based on patient specific information and historic information. This will allow for the multidirectional device to become fully automatic such that the directional changes of the hinge are being controlled and managed completely by the control system. The automated artificial intelligence control is based on at least one of a neural network, constraint program, fuzzy logic, classification, conventional artificial intelligence, symbolic manipulation, fuzzy set theory, evolutionary computation, cybernetics, data mining, approximate reasoning, derivative-free optimization, and soft computing.

In some embodiments, the operation apparatus is interchangeable between a first operation apparatus and a second operation apparatus, wherein the first and second operation apparatus comprise any of the following to be housed within the operation chamber: a vascular access apparatus that allows passing a guide wire into a target vessel with a special use in TIPS procedure, an intraluminal access system comprising a 10 mm needle that allows passing the guide wire and a semiflexible steel stylet into the lumen of a target with a special use in percutaneous nephrostomy, cholangiography and abscess drainage, a fiducial and brachytherapy marker placement system comprising an 18 mm needle that allows inserting fiducial marker placement in the organ of target, a radiofrequency ablation apparatus having a needle that allows passing the radiofrequency needle to reach a target organ, a fiberoptic scope system including a fiberoptic tube and a small camera that allows visualization and video recording during endovascular and endoluminal procedures, and a fiberoptic and camera visualization apparatus.

The present invention aims to provide a more efficient, maneuverable and precise device for performing image-guided percutaneous biopsy and interventional procedures. This fully automated, electronically navigated, artificial intelligence compatible device is capable to perform a variety of procedures and biopsies with more precision and less costs, and increases the safety and comfort of a patient. The present invention decreases the need for sedation and anesthesia of a patient and provides more patient adherence and satisfaction. Additionally, the multidirectional device is configured to decrease radiation exposure to the patients and personnel and decreases bodily fluid exposure hazards to the performing physician and personnel. The capability of being navigated remotely eliminates the need for patient transfer or physicians traveling for the purpose of image guided percutaneous biopsy and interventional procedures. This invention comprises a ball and socket hinge that connects the operation apparatus to the housing controls and permits incremental adjustments along three axes, such as inward, lateral and angular movement, and six degrees of freedom. All the functions can be controlled automatically by a joystick remote controller or can be navigated electronically by utilizing artificial intelligence and autopilot navigation if desired.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A multidirectional device for percutaneous procedures, comprising:
an operation chamber affixed to a sheath via a hinge;
wherein the sheath is an elongated tube;
wherein the operation chamber comprises a hollow body having an open first end configured to receive an operation apparatus and a second end affixed to the sheath;
a hinge system operably connected to the operation chamber, wherein the hinge system is configured to modify a path of the operation apparatus by controlling movement of the operation chamber and the operation apparatus during a surgical intervention.

2. The multidirectional device for percutaneous procedures of claim 1, further comprising the operation apparatus removably disposed within the operation chamber.

3. The multidirectional device for percutaneous procedures of claim 2, wherein the operation apparatus comprises a biopsy apparatus with a cutting blade configured to remove a tissue sample from a target tissue and a biopsy reservoir configured to store the tissue sample.

4. The multidirectional device for percutaneous procedures of claim 2, wherein the operation apparatus comprises a first operation apparatus interchangeable with a second operation apparatus.

5. The multidirectional device for percutaneous procedures of claim 1, wherein the hinge is formed by a ball disposed on the operation chamber and a socket disposed on the sheath.

6. The multidirectional device for percutaneous procedures of claim 5, wherein the hinge system comprises a first cord connected to the ball and passes through a socket cord aperture, wherein the first cord traverses along a length of the sheath until it reaches a hinge control disposed in a housing affixed to the sheath.

7. The multidirectional device for percutaneous procedures of claim 6, wherein the hinge control is a cogwheel scroll disposed within a cogwheel groove on the housing and the first cord is connected to an axel thereof such that as the cogwheel scroll is rotated, a length of the first cord is adjusted thereby rotating the ball.

8. The multidirectional device for percutaneous procedures of claim 6, further comprising a cord harness positioned along an interior side of the sheath and configured to secure the first cord in position to prevent cord movement.

9. The multidirectional device for percutaneous procedures of claim 5, wherein the hinge system comprises:
a first cord, a second cord, a third cord, and a fourth cord, each connected to and evenly distributed about the ball;
a first socket cord aperture, second socket cord aperture, third socket cord aperture, and fourth socket cord aperture, each within the socket and evenly distributed thereabout;
wherein each cord passes through a corresponding aperture such that the first cord originates from an opposing side of the first socket cord aperture, the second cord originates from an opposing side of the second socket cord aperture, the third cord passes originates from an opposing side of the third socket cord aperture, and the fourth cord originates from an opposing side of the fourth socket cord aperture.

10. The multidirectional device for percutaneous procedures of claim 1, further comprising:
a hinge lock system configured to prevent rotation of the hinge, wherein the hinge is a ball and socket;
the hinge lock system having a hinge lock and a hinge release button;
wherein the hinge lock is configured to physically couple the ball and socket so as to prevent relative rotation about the hinge;
the hinge release button configured to longitudinally move the hinge lock along the sheath.

11. The multidirectional device for percutaneous procedures of claim 10, wherein the hinge lock comprises a curved member that is operably connected to the hinge release button, wherein a terminating end of the hinge lock comprises a flexible tip configured to penetrate the ball aperture and the socket aperture.

12. The multidirectional device for percutaneous procedures of claim 10, wherein the hinge release button is movable along an L-shaped track disposed on a housing connected to the sheath, wherein the L-shaped track is adapted to move the hinge lock longitudinally along the sheath.

13. The multidirectional device for percutaneous procedures of claim 1, wherein an operation lever extends through an entire length of the sheath configured to slidably engage the operation apparatus.

14. The multidirectional device for percutaneous procedures of claim 1, further comprising a housing affixed to the sheath, wherein the housing includes a hinge control of the hinge system.

15. The multidirectional device for percutaneous procedures of claim 14, further comprising a main control system operably connected to the housing in order to perform all hinge movement and housing functions, wherein the main control system is a hand-held remote controller.

16. The multidirectional device for percutaneous procedures of claim 14, further comprising a robot for maneuvering the operation apparatus into the target tissue, the robot having a five-axis robot arm and an end-effector, the end-effector is attached to a distal end of the robot arm for the installation and insertion of the operation apparatus.

17. The multidirectional device for percutaneous procedures of claim 14, further comprising an automated artificial intelligence control configured to control the hinge system by determining pathway information based on patient specific information and historic information.

18. The multidirectional device for percutaneous procedures of claim 14, further comprising an actuation mechanism having a push button connected to a first end of an operation lever, wherein the push button extends from the housing and a second end of the operation lever is affixed to the operation apparatus, such that when the push button is depressed, the operation apparatus moves longitudinally outward from the operation chamber.

19. The multidirectional device for percutaneous procedures of claim 14, further comprising a housing spring disposed in the housing, wherein the housing spring is configured to bias the push button in an undepressed position.

20. The multidirectional device for percutaneous procedures of claim 19, further comprising a vacuum system having a piston operably connected to a cylinder, wherein a vacuum tube extends from the cylinder, through the sheath and terminating at the operation apparatus.
